(19) United States
(12) Patent Application Publication
(10) Pub. No.: US 2012/0052142 A1
(43) Pub. Date: Mar. 1, 2012

(54) BIOLOGICALLY ACTIVE COMPOSITIONS FROM Schisandra chinensis FOR TREATING COLORECTAL CANCER

(75) Inventor: John Noel GNABRE, Baltimore, MD (US)

(73) Assignee: Dr. Chang Naturals, LLC, South Deerfiled, MA (US)

(21) Appl. No.: 13/207,525

(22) Filed: Aug. 11, 2011

Related U.S. Application Data
(60) Provisional application No. 61/344,585, filed on Aug. 26, 2010.

(51) Int. Cl. A61K 36/79; A61P 35/00; A61K 31/09; A61K 31/357; A61K 31/216

(52) U.S. Cl. 424/777; 514/463; 514/549; 514/719

(57) ABSTRACT

A biologically active composition to treat colorectal cancer includes an extract of the plant Schisandra chinensis and a suitable pharmaceutical formulation. The extract can be at least one of gomisin A, angeloylgomisin H, deoxyschisandrin and gomisin N, showing activities against human tumor cell line HT-29 in the range of about 716 μM to about 43 μM. A suitable pharmaceutical formulation can be any one of a pill, a tablet, a capsule, a liquid solution, a liquid suspension, a powder, an intravenous solution, a gel and a suppository form. The extract is prepared by grinding the dried berries, extracting with hexane, extracting the marc with dichloromethane, extracting the dichloromethane soluble fraction with ethyl acetate, removing fatty materials in the dichloromethane-ethyl acetate soluble fraction with hexane, and fractionating the total hexane insoluble material extract using analytical high performance liquid chromatography equipped with automated repeat injection/collection cycles.

### CROSS REFERENCE TO RELATED APPLICATION

This application is based on, and claims the benefit of, U.S. Provisional Application No. 61/344,585, filed Aug. 26, 2010 with inventor John N. Gnabre, which is incorporated herein by reference.

### FIELD

Embodiments of the invention relate to biologically active compositions including an extract of the plant Schisandra chinensis and a pharmaceutical formulation for treating colorectal cancer. The compounds that exhibit inhibitory activities against colorectal cancer are gomisin A, angeloylgomisin H, deoxyschisandrin and gomisin N.

### BACKGROUND

The berry-bearing creeping vine Schisandra chinensis (Turcz.) Baill., (Schisandraceae or Magnoliaceae), commonly known as lemonwood, Chinese mock-barberry or “Chinese Magnolia”, is a popular medicinal plant endemic to the eastern regions of China, Japan, Korea and the far east region of Russia.

Today, research interests of Schisandra include diseases of the liver, digestive tract, lungs and kidneys. In Russia, Schisandra is a registered medicine for vision problems. In Chinese and Japanese traditional medicine, the dried ripe berries have been used for thousands of years for human ailments as varied as chronic cough, shortness of breath, inflammation, insomnia, diabetes, coronary heart disease, skin disorders, depression and menopausal symptoms. Schisandra species, especially, S. chinensis and S. sphenanthera, have been intensively investigated.

Lignans are major constituents of the Schisandra plants and are responsible for the myriad of activities ascribed to fruit extracts. The first dibenzocyclooctadiene lignan isolated from the fruit of Schisandra chinensis dated back to 1961 was termed schisandrin or gomisin in Japan. Recent advances in analytical techniques have enabled more comprehensive characterization of the lignans of these two Schisandra species. Over 50 constituents have been identified and categorized into five classes from type A to type E (Y. Lu and D.-F. Chen, J. Chromatography A 1216, 1980 (2009)). The most abundant class, the dibenzocyclooctadiene lignans (type A), makes up over 80% of the lignans in the fruit of S. chinensis. Several reports have associated Schisandra chinensis with various types of biological activities.

G. Zabrecky describes methods and formulations to treat chronic liver conditions using Schisandra chinensis as one of several plant components (U.S. Patent Application Publication 2010/0086627). However, Zabrecky does not describe any detail of the dibenzocyclooctadiene lignans and their activities against any cancer type.

A. W. Bartunek et al. provide compounds and methods for promoting cellular health and treating cancer, diabetes and glaucoma using Schisandra chinensis as one of several plant components (U.S. Pat. No. 7,794,758). Nevertheless, Bartunek et al. do not further describe whether colorectal cancer might have been treated and in what manner dibenzocyclooctadiene lignans may be helpful.

X. Hu publishes that gomisin N and related compounds may be used, in conjunction with known anticancer chemotherapeutic agents, to overcome P-glycoprotein-mediated multidrug resistant (MDR) cancer (U.S. Patent Application Publication 2005/0282910). According to Hu, the apparent mechanism is that the dibenzocyclooctadiene lignans inhibit P-glycoprotein, an ATP binding cassette (ABC) drug transporter from pumping the intracellular anticancer drug out of the cells. However, Hu does not show that any of the dibenzocyclooctadiene lignans may function as antitumor agents by themselves.

K. \check{S}mejkal et al. identifies a group of lignans for their potential against the BY-2 plant cell line (Planta Medica 2010: 76:1672-1677). However, \check{S}mejkal et al. do not find Gomisin N to be active against the colon cell line LoVo.

S. Y. Yim et al. states that gomisin N induces high apoptotic levels in hepatic carcinoma, especially in high concentrations (Molecular Medicine Reports 2:725-732, 2009). However, Yim et al. do not report any colorectal cancer activity from gomisin N.

H.-Y. Min et al. uses seven different cancer cell lines in identifying schisantherin C as a potential treatment of lung tumor (Bioorg. Med. Chem. Lett. 18:523-526 (2008)). Also, Min et al. seems to indicate that lignans with a methylenedioxy group in the A or B ring exhibit diminished activities, but lignans with a hydroxyl group at the C ring exhibit enhanced activities. In addition, the HCT-15 human colon tumor cell line used in Min et al. is known not to express the key tumor suppressor p53 protein as in the HT-29 human colon tumor cell line.

Therefore, there remains a need to investigate whether the dibenzocyclooctadiene lignans from Schisandra chinensis may be used for the treatment of colorectal cancer. In addition, there also remains a need to apply an analytical high performance liquid chromatography to efficiently isolate and purify the dibenzocyclooctadiene lignans.

### SUMMARY

In accordance with an embodiment of the invention, a biologically active composition to treat cancer includes an extract of Schisandra chinensis; and a pharmaceutical formulation, wherein the composition is useful for treating colorectal cancer.

In an embodiment of the invention, the extract comprises essentially at least one compound from the group consisting of gomisin A, angeloylgomisin H, deoxyschisandrin and gomisin N. In an embodiment of the invention, the extract comprises essentially gomisin N.

In an embodiment of the invention, the extract exhibits an inhibitory activity against a human colon HT-29 cell line. In an embodiment of the invention, the inhibitory activity is expressed as a half-maximal inhibitory concentration (IC₅₀) in the range of 716 μM to 43 μM.

In an embodiment of the invention, the pharmaceutical formulation is a member selected from the group consisting of a pill form, a tablet form, a capsule form, a liquid solution form, a liquid suspension form, a powder form, an intravenous solution form, a gel form and a suppository form.

In another embodiment of the invention, a method of preparing a biologically active composition from Schisandra chinensis includes: grinding dried berries of Schisandra chinensis into a powder of dried berries; extracting the powder of dried berries with hexane to form a marc having been washed with hexane, extracting the marc with dichloromethane to form a dichloromethane soluble fraction; extracting the dichloromethane soluble fraction with ethyl acetate to form a dichloromethane-ethyl acetate soluble fraction; removing fatty materials in the dichloromethane-ethyl acetate soluble fraction with hexane to yield a total hexane insoluble material extract; purifying from the total hexane insoluble material extract by using analytical high performance liquid chromatography equipped with automated repeat injection/collection cycles and guided by a biological activity against colon cancer to form an extract of Schisandra chinensis, and combining the extract of Schisandra chinensis with a pharmaceutical formulation.

In yet another embodiment of the invention, a method of treating colorectal cancer includes administering a biologically active composition of Schisandra chinensis to a person having symptoms of colorectal cancer, wherein the composition comprises an extract of Schisandra chinensis and a pharmaceutical formulation.

### BRIEF DESCRIPTION OF THE DRAWINGS

Embodiments of the invention will be more clearly understood by reference to the following detailed description of the embodiments and the accompanying drawings in which:

FIG. 1 is a diagram showing a scheme of assay-guided differential fractionation of the dried berries of S. chinensis. Powder of the berries was initially defatted with hexane and the marc was macerated with dichloromethane (DCM) to generate an active DCM extract. Further treatment of the DCM-exhausted marc by 50% methanol yielded inactive water-soluble materials. Plant extracts were tested stepwise for anti-proliferative activity against the human colorectal carcinoma cell line HT29.

FIG. 2 is an overlay of partial chromatograms of the total hexane insoluble material (THIM) extract from the first batch of S. chinensis, and the chemical structures of selected peaks. HPLC conditions are described in the Example section. Chromatograms were extracted at 220 nm. The chemical structure of each peak was determined by NMR/Mass Spectrometry.

FIG. 3 is a chromatogram of the THIM extract of Schisandra berries in the second batch, with peak identity. HPLC conditions are described in the Example section.

FIG. 4 is a comparison of the activities of the purified constituents of Schisandra berries on the human colorectal cancer cell line HT-29. Each point represents the average of six determinations from two separate experiments run in triplicates.

FIG. 5 is a diagram showing the chemical structures of the compounds isolated from S. chinensis and the key structural determinants influencing their anti-proliferative activities on the human colorectal cancer cell line HT-29.

FIG. 6 is a comparison of the chemical structures of (+) deoxyschisandrin and (−) gomisin N, with numbering system for the carbon atoms. The dotted line represents a plane of near-symmetry, broken only by the replacement of 2,3-dimethoxy in deoxyschisandrin with the 12,13-methylenedioxy group in gomisin N.

FIG. 7 is a view of gomisin N's X-ray crystal structure, with C9-C10-C15-C16 plane edge-on and C15 and C16 directly behind C10 and C9, respectively. The C1-C5, C16 aromatic ring is in the background, below H9α. Dihedral angles are shown for H6-H7 (H6-C7Me) and for H9-H8 (H9-C8Me).

### DETAILED DESCRIPTION OF THE EMBODIMENTS

Colon cancer is located in the large intestine, while rectal cancer is in the rectum. The difference between these two cancers is the location in the large intestine where the cancer occurs. Therefore, the term colorectal cancer is often used to refer to cancer in both locations. Colorectal cancer is third most common leading causes of cancer death in the United States. According to the “American Cancer Society Colorectal Cancer Facts and Figures 2011-2013” (Atlanta, American Cancer Society, 2011), in 2001, the incidence rates of colorectal cancer per 100,000 are about 57.2 among male, and about 42.5 among female. In comparison, the mortality rates are 21.2 among male, and 14.9 among female per 100,000. As a whole, there will be about 141,000 new cases and 49,000 deaths in 2011.

Colorectal cancer usually develops slowly over a period of 10 to 15 years, starting as a non-cancerous polyp on the lining of the colon or the rectum. Certain types of polyp, called adenocarcinoma, are likely to become cancerous. Once colorectal cancer forms, it can grow through the lining into the wall of the colon or the rectum. Cancer in the colorectal area may spread to nearby lymph nodes, or can be carried by blood vessels to liver and lungs, or it may spread in the abdominal cavity to other locations such as the ovary.

The common stages of colorectal cancer includes: Stage 0: when cancer is only on the innermost layer of the intestine; Stage I: when cancer is in the inner layer of the colon; Stage II: when cancer has spread through the muscle wall of the colon; Stage III: when cancer has spread to the lymph nodes; and Stage 1V: when cancer has spread to other organs. The most effective approach to treat colorectal cancer is early detection before symptoms develop by undergoing periodic colonoscopy or sigmoidoscopy when a person is 50 years or older, or has either a family history or personal history of colon cancer. The treatment options of colorectal cancer are surgery, radiation therapy and chemotherapy. 5-Fluorouracil, oxaliplatin and irinoteccan are commonly used chemotherapeutic agents for colorectal cancer.

In accordance with an embodiment of the invention, a biologically active composition to treat cancer includes an extract of Schisandra chinensis; and a pharmaceutical formulation, wherein the composition is useful for treating colorectal cancer.

In one embodiment, the extract comprises essentially at least one compound from gomisin A, angeloylgomisin H, deoxyschisandrin and gomisin N. In another embodiment of the invention, the extract comprises essentially gomisin N.

In an embodiment of the invention, the extract exhibits an inhibitory activity against a human colon HT-29 cell line. In an embodiment of the invention, the inhibitory activity is expressed as a half-maximal inhibitory concentration (IC₅₀) in the range of 716 μM to 43 μM.

In an embodiment of the invention, the pharmaceutical formulation may be utilized in conjunction with the extract of Schisandra chinensis to form a composition that is suitable for treating colorectal cancer. The pharmaceutical formulation can be in a pill form, a tablet form, a capsule form, a liquid solution form, a liquid suspension form, a powder form, an intravenous solution form, a gel form or a suppository form.

In another embodiment of the invention, a method of preparing a biologically active composition from Schisandra chinensis includes: grinding dried berries of Schisandra chinensis into a powder of dried berries; extracting the powder of dried berries with hexane to form a marc having been washed with hexane, extracting the marc with dichloromethane to form a dichloromethane soluble fraction; extracting the dichloromethane soluble fraction with ethyl acetate to form a dichloromethane-ethyl acetate soluble fraction; removing fatty materials in the dichloromethane-ethyl acetate soluble fraction with hexane to yield a total hexane insoluble material extract; purifying from the total hexane insoluble material extract by using analytical high performance liquid chromatography equipped with repeat injection/collection cycles and guided by a biological activity against colon cancer to form an extract of Schisandra chinensis, and combining the extract of Schisandra chinensis with a suitable pharmaceutical formulation.

In yet another embodiment of the invention, a method of treating colorectal cancer includes administering a biologically active composition of Schisandra chinensis to a person having symptoms of colorectal cancer, wherein the composition comprises an extract of Schisandra chinensis and a suitable pharmaceutical formulation.

Schisandra ripe berries have also been shown to be rich in phytoestrogens, minerals, vitamins and essential oils. These chemical contents may be responsible for the pungent, sour, sweet, salty and bitter taste, hence its Chinese denotation of w\check{u} wèi zi or “five flavor fruit”. Schisandra berries have been known in the U.S. since the 1950's, sold mostly in natural food stores in extract form. Until recently, all Schisandra berries in the U.S. were imported from China. Now U.S. grower, Dr. T. C. Chang, offers certified-organic Schisandra in several quick-frozen formats.

The comestible use of the fruit products and its potential anticancer properties prompted the current investigation as to whether the active constituents could be used to treat colorectal cancer. The robust MTS cell viability assay with the p53-mutant human colon HT-29 cell line served as an assay-guided screening tool for the purification of the active principles of the fruit extract (see further description in the Example section).

Compound purity is critical in the paradigm of drug discovery. Therefore, more and more analytical laboratories have been combining state-of-the-art automated analytical instrumentations with high throughput screening to achieve high efficiency in drug discovery. Thus, a number of analytical methods have been used to resolve the constituents of the extracts of Schisandra berries but with varying degrees of success. More recently, tandem HPLC/mass spectrometry and NMR was applied to the separation and characterization of a large number of Schisandra fruit components. However, very few analytical separation techniques have been able to generate substantial amounts of pure compounds for pharmacological evaluation.

The current study takes advantage of the progress in the automation of the separation techniques enabled by the flexibility of analytical HPLC interfaces. Thus, pure dibenzocyclooctadiene lignans contained in Schisandra berry crude extract were separated in a single run on a reversed phase analytical HPLC due to the high number of theoretical plates provided by the analytical scale column. Conditions were optimized in order to achieve a reproducible separation over a period of 48-96 hours, allowing separation of 1-20 milligrams of pure compounds by multiple consecutive injection/collection cycles. In this study, a cost-effective and highly efficient strategy superior to the standard semi-preparative HPLC is being explored.

The dichloromethane (DCM) extract of Schisandra dry berries were initially compared by thin-layer chromatography (TLC) to establish whether the season of collection had some influence on the contents of active principles. The results revealed no significant difference. However, the fall batch appeared to be slightly richer in plant constituents than the winter batch, as assessed by TLC.

The activity of the two batches was then assessed on three common cancer cell lines at a concentration varying from 1 to 100 μg/mL to determine whether the active constituents would exhibit any specificity against a particular type of cancer cell. The results consistently revealed that the ingredients of the DCM extracts of both the two batches had strong activity against the human colon HT-29, but no detectable activity against the erythroleukemia K562 cell line, and only weak activity against the human breast MCF-7 cell line. There was No Significant Difference Between the Two Batches. Based on these results, the HT-29 cell line was used as a target for the fractionation and isolation of the anti-proliferative activity of the two batches of Schisandra berries.

### Differential Fractionation of the Powder of Schisandra Berries

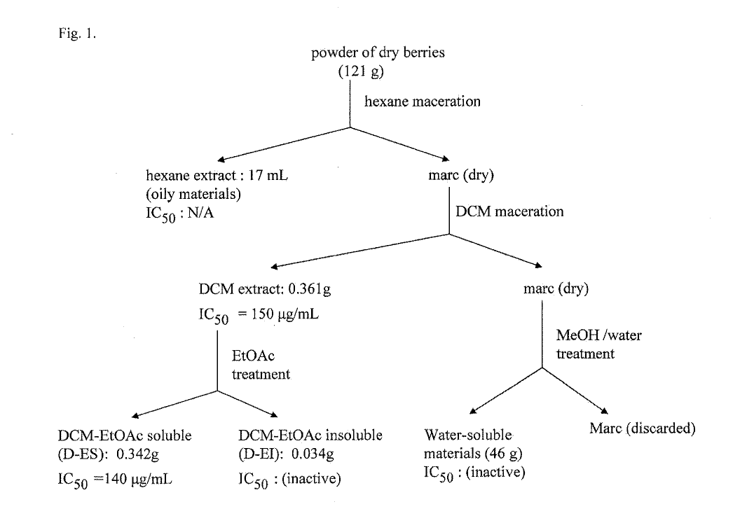

Powder of dried Schisandra berries was macerated with hexane and the marc was then extracted by bioassay-guided differential fractionation (FIG. 1).

The dichloromethane/ethyl acetate (DCM-EtOAc) soluble materials (D-ES) were dried and further defatted with hexane to yield a working extract referred to as the total hexane insoluble materials (THIM) which was used in the HPLC studies. All extracts were analyzed by TLC and stepwise tested for inhibition of human carcinoma HT-29 cell growth as described in the Example section. Although thoroughly defatted, the half-maximal inhibitory concentration, IC₅₀, of the THIM extract (136 μg/mL) was not significantly different from that of the D-ES extract (140 μg/mL).

### Fractionation by HPLC of the First Batch of the THIM Extract

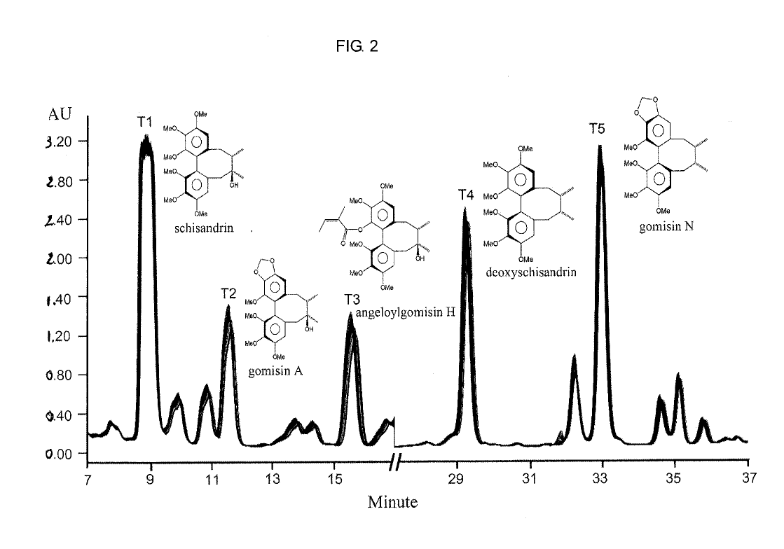

The separation of the first batch of the THIM Extract was performed using a gradient of 0.1% acetic acid in acetonitrile (solvent B) and 0.1% acetic acid in water (solvent A) and the flow rate set at 1.0 ml/min. The elution gradient was changed from 55% solvent B to 100% B over 50 minutes with the non-linear gradient curve 7. The column was cleaned 15 minutes with 100% solvent B and re-equilibrated for 10 minutes before the next injection. About 20 mg of the THIM extract was dissolved in 4 mL of acetone/hexane to yield a concentration of 5 mg/mL. The injection volume was fixed at 30 μL, resulting in about 150 μg of sample injected in the column for each injection. The sample was injected about 40 consecutive times and the collection was achieved using a Waters Fraction Collector II. A blank run preceded each collection procedure. The automated injection/collection cycles were repeated over a 48-hour period with the partial overlay shown in FIG. 2. All fractions were dried, weighed, and analyzed by TLC with cerium sulfate charring and the targeted peaks (T1 to T5) were tested for biological activity.

The full chromatogram of FIG. 2 shows a peak cluster eluting in the first 8 minutes but was devoid of detectable biological activity. However, a second cluster eluting from the 17^{th} to the 28^{th} minute was active but much less than the parent THIM extract. Furthermore, the second cluster was still relatively complex, and was without any outstanding peak. As a result, the second cluster did not under further separation.

The chemical structure was determined by NMR/Mass spectrometry. The degree of purity of each peak was determined by tandem HPLC/NMR under the conditions described in the Example section and was found to be >90%. The overlay of the chromatograms in FIG. 2 reflects reproducibility of the analytical reverse phase HPLC method that was used to purify the complex plant extract. The automation of injection/collection cycles repeated over several days with this reproducibility was made possible by the flexibility and versatility of the chromatography manager Empower 2 of the HPLC system. FIG. 2 is therefore the evidence that the strategy of purification of complex extracts on analytical reverse phase C18 is efficient and reliable.

### Fractionation by HPLC of the Second Batch of the THIM Extract

With the second batch of the THIM extract, samples were fractionated on the same HPLC instrument as previously described for the first batch but with different conditions. In the fractionation of the second batch, the HPLC conditions were optimized using the observations acquired from the fractionation conditions of the first batch of the THIM extract. For instance, the introduction of hexane in the eluent was helpful in eluting inactive peaks more quickly, and thus the peaks of interest can be resolved within 45 minutes before column reconditioning.

Samples were eluted at 1 ml/min with a gradient of 0.1% glacial acetic acid in water (solvent A), 0.1% glacial acetic acid in MeCN (solvent B), and 100% hexane (solvent C) for post separation column cleaning. The gradient was as follows: from 55:45:0 A/B/C to 15:85:0 over 35 minutes with the non-linear gradient curve 7, immediately the eluent composition was changed to 0:100:0; at minute 36 it was immediately changed to 0:0:100 (column cleaning with hexane); at minute 38, it was immediately changed to 0:100:0, isocratic elution until minute 46, then linear gradient to 55:45:0 at minute 47. Hexane shows very limited solubility (5-10%) in MeCN (acetonitrile) at room temperature, therefore, its use is not common on RP C18 columns. In this step, the addition of hexane to the HPLC separation procedure was preferred over adding another cleaning step to the extract preparation since the low solubility was sufficient to remove remaining traces of hexane from the column during 100% MeCN re-conditioning (min 38-46). Hexane also happened to be efficient in removing unwanted materials such as chlorophyll or fatty acids strongly retained by the RP C18 stationary phase. Immediate eluent composition changes were achieved using gradient curve 1. Re-equilibration time was 10 minutes, and each sample set started with a blank injection. The plant extract was dissolved in the starting mobile phase (55:45:0, A/B/C) and 30 μl of saturated solution was the typical injection volume. These improved conditions along with the automated injection/collection cycles enabled to achieve a separation of 3-18 milligrams of pure compounds over a 96-hour period without a significant decrease in the purity of the targeted peaks (degree of purity>95%) as assessed by tandem HPLC/NMR under the conditions described in the experimental section for the NMR.

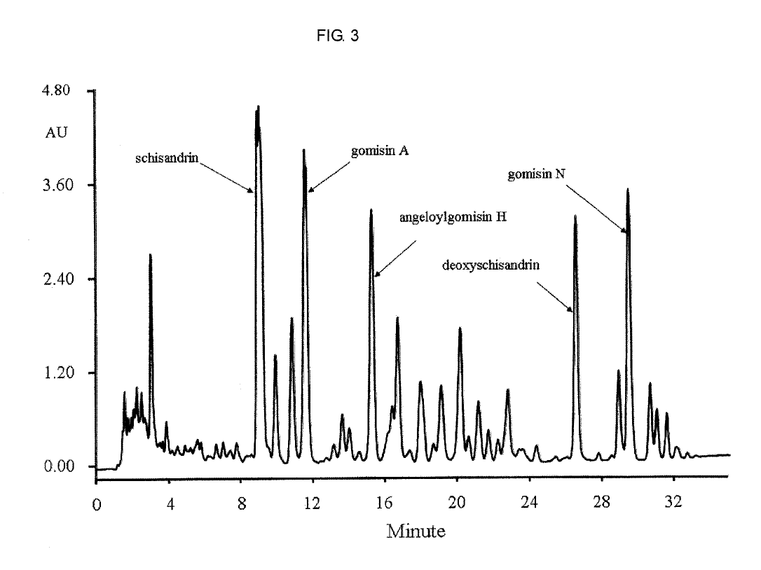

The chromatogram (FIG. 3) of the second batch shows a slight increase in the overall peak number, as compared to the full chromatogram of the first batch, which may result from the improved HPLC conditions. The fractions were dried, weighed and analyzed by TLC with cerium sulfate charring.

As a comparison, the amount of gomisin N in the first batch run (in the non-optimized conditions) was 1.2 mg over a 48-hour period. Using the same non-optimized conditions, the amount of gomisin N would have been 2.4 mg over a 96-hour period (a 2-fold increase, assuming that the relationship is linear). But because of the optimization of the running conditions for the second batch fractionation, it was found that the amount of gomisin N (23 mg) was actually increased by nearly 10-fold. Therefore, the optimized conditions did significantly improve the fractionation of the second batch. The multiple repeat-injection method of analytical HPLC was a superior alternative method to the standard semi-preparative HPLC techniques which is often used for most separation of complex mixtures.

As described in FIG. 2, the chromatogram of the second batch was also extracted at 220 nm and the compound identifications were achieved by NMR and Mass Spectrometry. Tandem HPLC/NMR analysis confirmed the targeted peaks to be >95% pure. The peak cluster eluting from the 17^{th} to the 25^{th} minute was twice less active than the parent THIM extract, and therefore was not further separated.

Table 1 summarizes the results of the HPLC separation of the two batches of the THIM extracts. Although conditions were optimized in the second batch, there was no significant difference in the peak numbers and their elution times. As observed in FIG. 1, the DCM extract exhibits an IC₅₀ against the human colorectal cell line HT-29 at 150 μg/ml and the D-ES extract at 140 μg/ml. In comparison, the THIM extract exhibits an IC₅₀ against HT-29 at 136 μg/ml (Table 1). While schisandrin does not show any activity against HT-29, the four dibenzocyclooctadiene lignans show interesting anti-colorectal cancer activities against HT-29: gomisin A at 300 μg/ml, angeloylgomisin H at 348 μg/ml, deoxyschisandrin at 140 μg/ml and gomisin N at 17 μg/ml (Table 1).

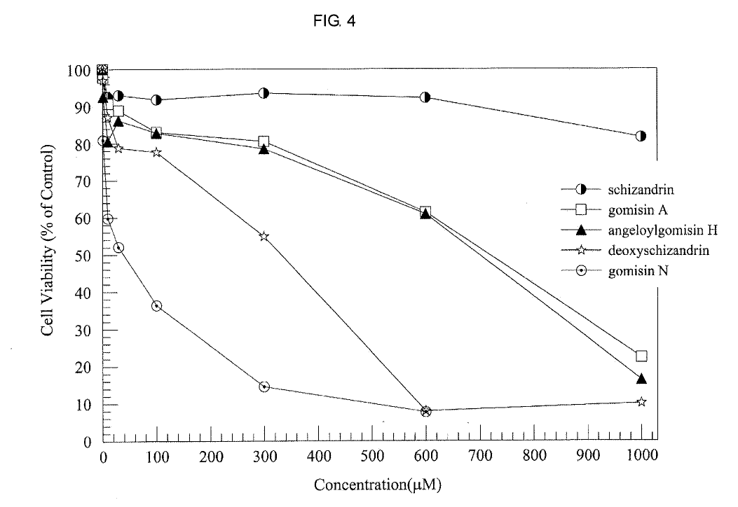

Once the structures of the compounds were identified, their anti-proliferative activity in the bioassay was compared on a molar basis, as shown in FIG. 4.

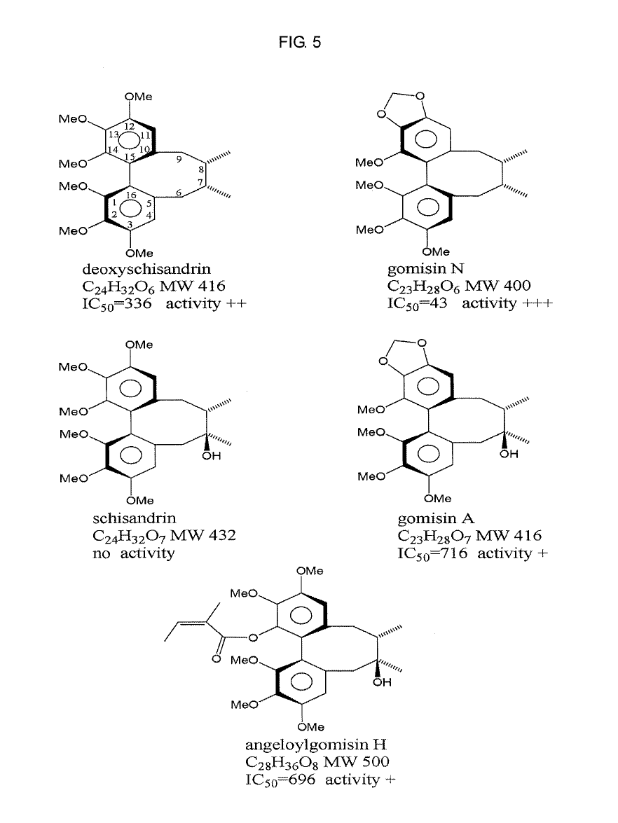

The concentrations inhibiting cell growth by 50% (IC₅₀) were determined from the dose-response curves and were the following: gomisin A, 716 μM; angeloylgomisin H, 696 μM; deoxyschisandrin, 336 μM; and gomisin N, 43 μM (FIG. 5). Schisandrin was devoid of detectable biological activity.

TABLE 1
Summary of Two Analytical Reverse Phase HPLC Fractionations of
the Extract from the Berries of S. chinensis.

| | Initial HPLC | | Optimized HPLC | | |
|---|---|---|---|---|---|
| | Fractionation | | Fractionation | | |
| | 48 Hr Period | | 96 Hr Period | | |
| | Elution | Dry | Elution | Dry | Activity ^{a} |
| | Time | Weight | Time | Weight | IC₅₀ |
| Compound | (min) | (mg) | (min) | (mg) | (μg/ml)^{b} |
| THIM Extract | | | | | 136 |
| Schisandrin | 9.0 | 3.1 | 9.0 | 17.8 | >1000 |
| Gomisin A | 11.5 | 1.6 | 11.5 | 5.4 | 300 |
| Angeloylgomisin H | 15.5 | 1.1 | 15.5 | 3.7 | 348 |
| Deoxyschisandrin | 29.5 | 1.4 | 26.5 | 2.7 | 140 |
| Gomisin N | 33.0 | 1.2 | 29.5 | 23.0 | 17 |

^{a} Activity was determined in the human colorectal carcinoma cell line HT29 using the MTS cell viability assay.
^{b}IC₅₀ values were derived from the sigmoidal dose-response curves. Values are the average of two independent experiments performed in triplicate.

### Structure-Activity Relationships of the Compounds

The mechanism underlying the anti-proliferative activity of these lignans on their molecular target in human colorectal cancer is unknown. The current results were obtained in a single cultured cancer cell line (HT-29) but reveal consistent structural determinants for the activity of these molecules, as shown in FIG. 5.

A comparison of schisandrin with deoxyschisandrin shows that the 7-OH group in schisandrin reduces anticancer activity. A similar phenomenon is also observed when gomisin A is compared with gomisin N, in that the 7-OH group in gomisin A also decreases anticancer activity. A comparison of gomisin A with schisandrin reveals that the methylenedioxy group between C12 and C13 in gomisin A enhances activity against colorectal cancer. This observation also is true when gomisin N is compared with deoxyschisandrin, in that the methylenedioxy group between C12 and C13 in gomisin N also enhances activity against colorectal cancer. In contrast, Min et al. seems to conclude that the presence of the 7-OH group apparently enhances activity, while the presence of the methylenedioxy group diminishes activity.

Gomisin N, the most active of these five isolated compounds from Schisandra chinensis, has both of these activity-enhancing determinants. However, gomisin N has the opposite twist of the biphenyl ring system (S-biphenyl configuration), which changes the shape of the molecule than the other isolated compounds. In comparison, Min et al. reports that gomisin N with the S-biphenyl configuration is more active against a number of cancer cells than wuweizisu B, which has the R-biphenyl configuration. However, the Min study utilized the HCT-15 colorectal carcinoma cell line that lacks several chromosomes and does not express the key tumor suppressor p53 protein that is characteristic of the HT-29 cell line.

Angeloylgomisin H differs from the inactive schisandrin only by having an angeloyl group at C14, yet it exhibits moderate activity against colorectal cancer.

### NMR Study of the Most Active Compounds

The two most active fractions T4 and T5 (see FIG. 2), were identified as deoxyschisandrin and gomisin N, respectively, and were studied in more details by 1D and 2D NMR methods than previously reported. The 600-MHz ¹H NMR spectrum of T4 showed six resolved methoxy singlets (four near 3.88 ppm and two near 3.59 ppm); two methyl doublets in the upfield region (0.7-1.0 ppm); two aromatic singlets near 6.5 ppm; and two ABX systems with the AB portions in the 2.0-2.6 ppm region and the X portions near 1.8 ppm. The ¹H NMR spectrum of T5 was similar, but with only four methoxy peaks and with a new AB pattern near 5.9 ppm with J_{ab}=1.5 Hz. This indicates that the two adjacent methoxy groups in T4 have been replaced with a methylenedioxy (O—CH₂—O) group forming a 5-membered ring in T5. Aside from this particular difference, there are striking similarities between T4 and T5 in the ¹H NMR and ¹³C NMR chemical shifts, and in the ¹H coupling patterns and J values.

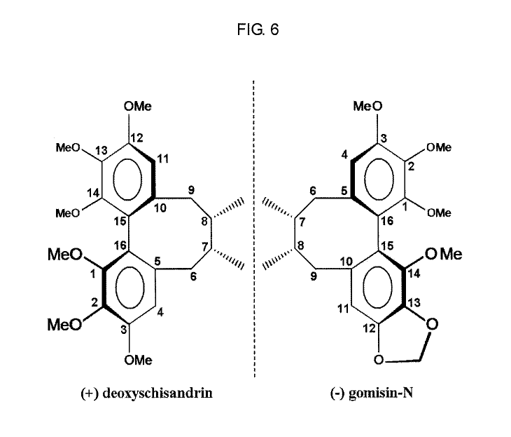

Two-dimensional DQF-COSY, HSQC and HMBC spectra at 600 MHz ¹H NMR frequency proved the covalent structure of deoxyschisandrin (T4) and gomisin-N (T5). Molecular modeling and prediction of the 3-bond H—H and C—H coupling constants confirmed the stereochemistry of the methyl groups in the 8-membered ring as well as the biphenyl “twist” stereochemistry. Comparison of the structures of (+) deoxyschisandrin and (−) gomisin-N (FIG. 6) shows that there is a near mirror-image symmetry, with the only difference being the replacement of the 2-OMe and 3-OMe groups of (+) deoxyschisandrin with the 12,13-O—CH₂—O ring of (−) gomisin-N.

This symmetry is reflected in the ¹H shifts and J-coupling patterns (Table 2) as well as in the ¹³C shifts (Table 3), provided that the reversed numbering system of gomisin N is accounted for in the comparison (e.g., C1 of deoxyschisandrin corresponds to C14 of gomisin N in FIG. 6).

Published ¹H NMR data on deoxyschisandrin at 100 MHz (Y. Ikeya, H. Taguchi, I. Yoshioka, H. Kobayashi, Chem. Pharm. Bull. 27 (1979), 2695) and 300 MHz (S.-J. Jiang, Y.-H. Wang, D.-F. Chen, Zhongguo Tianran Yaowu 3 (2005) 78) only reported chemical shift values. Only two methoxy chemical shifts are reported and they are not assigned to specific positions. A single chemical shift is given for protons 9α and 9β, and for protons 4 and 11. From the 300 MHz data, the 6α and 6β protons were assigned as a single shift, and the H6/H9 and H7-Me/H8-Me assignments are reversed.

Published ¹³C NMR data on deoxyschisandrin (S.-J. Jiang et al.) closely matched the data in Table 3, except that the C2/C13 signals are reversed, and the methoxy carbons are not specifically assigned.

TABLE 2
¹H Chemical Shift Assignments at 600 MHz NMR for Gomisin N and
Deoxyschisandrin in CDCl₃. Chemical shifts are relative to tetra-
methylsilane at 0 ppm. Protons in the CH₂ groups are designated
as α for being below the plane of the molecule as drawn in
FIG. 6 and as β for being above the plane. Assignments
were confirmed by 2D HSQC, DQF-COSY and HMBC.

| Gomisin N | | | Deoxyschisandrin | | |
|---|---|---|---|---|---|
| Position | ¹H ppm | Multiplicity | Multiplicity | ¹H ppm | Position |
| 1-OMe | 3.544 | s, 3H | s, 3H | 3.585 | 14-OMe |
| 2-OMe | 3.893 | s, 3H | s, 3H | 3.884 | 13-OMe |
| 3-OMe | 3.881 | s, 3H | s, 3H | 3.888 | 12-OMe |
| 4 | 6.549 | s, 1H | s, 1H | 6.542 | 11 |
| 6α | 2.568 | dd, 1H, 13.6, | dd, 1H, 13.5, | 2.581 | 9α |
| | | 7.3 | 7.4 | | |
| 6β | 2.521 | dd, 1H, 13.6, | dd, 1H, 13.5, | 2.502 | 9β |
| | | 2.2 | 1.9 | | |
| 7 | 1.888 | m, 1H | m, 1H | 1.901 | 8 |
| 7-Me | 0.732 | d, 3H, 7.2 | d, 3H, 7.2 | 0.739 | 8-Me |
| 8 | 1.778 | m, 1H | m, 1H | 1.811 | 7 |
| 8-Me | 0.968 | d, 3H, 7.2 | d, 3H, 7.2 | 1.001 | 7-Me |
| 9α | 2.223 | dd, 1H, 13.3, | dd, 1H, 13.3, | 2.278 | 6α |
| | | 9.5 | 9.6 | | |
| 9β | 2.020 | dd, 1H, 13.3, | dd, 1H, 13.3, | 2.055 | 6 β |
| | | 1.4^{a} | 1.3^{a} | | |
| 11 | 6.476 | s, 1H | s, 1H | 6.534 | 4 |
| O—CH₂—O | 5.941 | d, 1H, 1.5 | s, 3H | 3.899 | 3-OMe |
| O—CH₂—O | 5.947 | d, 1H, 1.5 | s, 3H | 3.873 | 2-OMe |
| 14-OMe | 3.816 | s, 3H | s, 3H | 3.589 | 1-OMe |

^{a}J value estimated from line broadening.

TABLE 3
¹³C Chemical Shift Assignments at 600 MHz for Gomisin N and
Deoxyschisandrin in CDCl₃. Chemical shifts are relative to
solvent CDCl₃ at 77.0 ppm. Assignments were confirmed
by 2D HSQC, DQF-COSY and HMBC.

| Gomisin N | | | Deoxyschisandrin | | |
|---|---|---|---|---|---|
| Position | Lit ^{a} | ¹³C ppm | ¹³C ppm | Lit ^{b} | Position |
| 1 | 151.6 | 151.59 | 151.50 | 151.62 | 14 |
| 1-OMe | 60.5 | 60.55 | 60.54 | 60.74 | 14-OMe |
| 2 | 140.0 | 139.99 | 140.33 | 139.97 | 13 |
| 2-OMe | 61.0 | 61.01 | 60.95 | 61.17 | 13-OMe |
| 3 | 151.5 | 151.52 | 151.56 | 153.09 | 12 |
| 3-OMe | 55.8 | 55.86 | 55.87 | 56.12 | 12-OMe |
| 4 | 110.6 | 110.58 | 110.43 | 110.74 | 11 |
| 5 | 134.1 | 134.08 | 133.90 | 134.17 | 10 |
| 6 | 39.1 | 39.10 | 39.13 | 39.37 | 9 |
| 7 | 33.5 | 33.53 | 33.74 | 33.98 | 8 |
| 7-Me | 12.8 | 12.82 | 12.65 | 12.88 | 8-Me |
| 8 | 40.7 | 40.69 | 40.75 | 41.00 | 7 |
| 8-Me | 21.6 | 21.56 | 21.81 | 21.99 | 7-Me |
| 9 | 35.4 | 35.48 | 35.57 | 35.82 | 6 |
| 10 | 137.8 | 137.81 | 139.41 | 139.40 | 5 |
| 11 | 102.9 | 102.94 | 107.12 | 107.43 | 4 |
| 12 | 148.6 | 148.62 | 152.83 | 153.09 | 3 |
| O—CH2—O | 100.7 | 100.71 | 55.90 | 56.12 | 3-OMe |
| 13 | 134.50 | 134.51 | 139.69 | 140.32 | 2 |
| — | — | — | 60.93 | 61.17 | 2-OMe |
| 14 | 141.0 | 141.00 | 151.37 | 151.75 | 1 |
| 14-OMe | 59.6 | 59.63 | 60.54 | 60.74 | 1-OMe |
| 15 | 121.3 | 121.30 | 122.29 | 122.57 | 16 |
| 16 | 123.2 | 123.25 | 123.34 | 123.62 | 15 |

^{a} S.-J. Jiang, Y.-H. Wang, D.-F. Chen, Zhongguo Tianran Yaowu 3 (2005) 78.
^{b} H. Iwata, Y. Tezuka, S. Kadota, A. Hiratsuka, T. Watabe, Drug Metab. Dispos. 32 (2004) 1351.

Gomisin N has better structural characterization in the literature. The reported 100 MHz proton data (Y. Ikeya, H. Taguchi, I. Yoshioka, H. Kobayashi, Chem. Pharm. Bull. 27 (1979), 2695)) show only three methoxy signals, without assignment, a single shift for H6α/H6β and for H7/H8, and the H4 and H11 assignments are backwards. Data reported at 400 MHz (H. Tang, Y. Yi, X. Yao, Q. Xu, S. Zhang, P. Sun, Zhongguo Haiyang Yaowu 21 (2002) 11) correctly differentiate the H7 and H8 peaks, but the H9α and H9β assignments are reversed. In both cases, the J couplings are reported only for the doublet methyl groups and for H9α and H9β. In particular, the position 9 coupling data match that in the application very well (Table 2) (13.5/8 Hz and 13.5/1 Hz (Y. Ikeya et al.); 13.2/9.2 Hz and 13.2 br. d. (H. Tang et al.).

A more recent report of 400 MHz ¹H NMR data shows J couplings only for the doublet methyl groups, whose assignments are reversed (H. Iwata, Y. Tezuka, S. Kadota, A. Hiratsuka, T. Watabe, Drug Metab. Dispos. 32 (2004) 1351). NMR data at 500 MHz ¹H frequency were reported in CD₃OD for deoxyschisandrin and in CDCl₃ for gomisin N (S.-M. Seo, H.-J. Lee, Y. Park, M. K. Lee, J.-I. Park, K.-H. Pails, Nat. Prod. Sci. 10 (2004) 104).

The deoxyschisandrin ¹³C shifts match the data in this subject application (average Δδ=0.25 ppm, std. dev. 0.03), except for the reversed assignments of C2/13 and C1/14. The J coupling values for the deoxyschisandrin AB(X) systems are similar to the data of the application, but the assignments are not stereospecific (“6a” 13.5/2.0 Hz and “6b” 13.5/7.5 Hz). An X-ray crystal structure is available for gomisin N (J. Marek, J. Slanina, Acta Crystallogr., Sect. C 54 (1988) 1548 (Cambridge Crystallographic Database POVM1W).

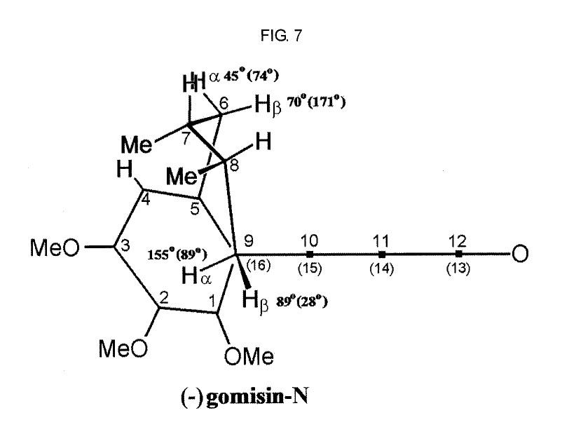

The stereospecific assignments are based on the X-ray structure of gomisin N and an energy-minimized model of deoxyschisandrin. FIG. 7 shows a view of the gomisin N structure with C16 directly behind C9 and C15 directly behind C10. The calculated deoxyschisandrin structure is essentially the mirror image of this structure, with corresponding numbering as shown in Tables 3 and 4.

Dihedral angles from the H7 proton to H6α/H6β and from the H8 proton to H9α/H9β of gomisin N are shown, with nearly orthogonal dihedrals corresponding to the small vicinal H—H couplings observed (89° angle, J_{8-9β} not resolved; 70° angle, J_{7-6β} 2.2 Hz). Similar results were seen with the calculated deoxyschisandrin structure (91° angle, J_{7-6β} not resolved; 68° angle, J_{8-9β} 1.9 Hz). Dihedral angles for 3-bond C—H couplings correspond well with the observed intensities of the HMBC crosspeaks. For gomisin N, the H9β-C8Me(28°) and H6β-C7Me(171°) crosspeaks are clearly observed but the H9α-C8Me(89°) and H6α-C7Me(74°) are missing.

Likewise for deoxyschisandrin, the H6β-C7Me(21°) and H9β-C8Me(179°) are strong but the H6α-C7Me(92°) and H9α-C8Me(71°) are missing. In every case (16 selected HMBC crosspeaks for each compound), the relative intensity of gomisin N HMBC crosspeaks correlated very closely with those of the pseudosymmetry-related deoxyschisandrin crosspeaks. The strong correlations between dihedral angle and ³J_{HH} or HMBC crosspeak intensity confirm the relative stereochemistry of the two methyl-bearing carbons (C7 and C8) and the biphenyl twist (C5-C16-C15-C10) in both compounds.

The results of the current study show that many of the active constituents of the Schisandra fruit extract can be isolated by analytical HPLC in milligram amounts, enabling both structure elucidation and pharmacological testing. For years, analytical HPLC has been the reference method for the quantitative and qualitative analysis of the contents of complex mixtures. This method has been useful in purifying most of the constituents of Schisandra berries [4]. Under the experimental conditions developed in this study, 1-20 milligrams of pure dibenzocyclooctadiene lignans were isolated from the Schisandra fruit extracts by unattended operating reversed phase HPLC for continuous 2-4 days. Because of the increased resolution of this analytical technique, the method described herein may supersede standard semi-preparative methods for the separation of complex mixtures.

NMR methods and spectrometers are constantly improving, yielding higher resolution and more precise correlations. The 600 MHz data for gomisin-N and deoxyschisandrin clarify some errors and disagreements in the literature and add new information on dihedral angles. Both measured H—H coupling constants and HMBC crosspeak intensities, which are related directly to H—C coupling constants, can be used to test conformation and stereochemistry in structural models. The solution NMR data are consistent with the X-ray crystal structure of gomisin N, as well as with the calculated structure of deoxyschisandrin. In addition, ¹H chemical shifts could be stereospecifically assigned for the two aliphatic methylene groups of each compound.

### EXAMPLES

### Plant Materials

Frozen ripe berries of Schisandra chinensis were provided by Dr. Chang Naturals, LLC of P.O. Box 191, South Deerfield, Mass. 01373, U.S.A. Two batches of the plant specimens were collected and shipped at different seasons. The first batch was received in October 2007 and the second batch in February 2009. The berries were shipped frozen, and upon arrival, were immediately thawed and placed in a 45° C. oven for several days until they were dry. The materials were then ground into a powder in a Coffee Mill blender, and the dibenzocyclooctadiene lignans were extracted following the scheme outlined in FIG. 1. All fractions generated by subsequent HPLC studies were dried in a SpeedVac (Savant Instruments Inc., Holbrook, N.Y.), weighed and monitored by silica gel thin-layer chromatography (TLC) with cerium sulfate charring [2% CeSO₄ (w/v) in 5.6% H₂SO₄ (v/v)]. For testing in cell culture, all dried test materials were solubilized at 100 mg/mL or at 100 mM in dimethylsulfoxide (DMSO) to give stock solutions which were stored at −20° C.

### HPLC Reagents and Instruments

Acetonitrile (MeCN) and glacial acetic acid were purchased from J. T. Baker (Phillipsburg, N.J., USA). HPLC grade water was purchased from Fisher Scientific (Fair Lawn, N.J., USA). The fractionation of the plant extracts was performed using a Waters 2695 Separations Module (Waters Associates, Milford, Mass.) equipped with a Waters 2996 photodiode array detector (PDA) a Waters fraction collector II, and an Atlantis dC18 reversed phase column (4.6×150 mm, 3 μm particle size, Part No. 18800-1342, Waters) maintained at 35° C. The system was controlled by an Empower 2 chromatography manager (Waters). The PDA detector was operated between 200 and 400 nm, and qualitative profile chromatograms were extracted at 220 nm

### Cell Lines

Erythroleukemia K562, human colon HT-29 and breast MCF-7 cancer cell lines were obtained from the American Type Culture Collection (Manassas, Va., USA). All cells were maintained in Dulbecco's modified Eagle's medium (DMEM) supplemented with 10% fetal bovine serum, penicillin (100 U ml⁻¹) and streptomycin (100 U ml⁻¹). Cells were incubated at 37° C. in a 5% CO₂ humidified atmosphere.

### MTS Cell Viability Assay

Cells were seeded at a density of 1.5×10⁴ cells/well on a 96-well plate. When the cells reached 60 to 70% confluency, the growth medium was aspirated and the wells were rinsed with pre-warmed PBS. Test compounds at various concentrations or 1% DMSO (vehicle control) were then added and the plates were incubated for 72 h. After incubation, 40 μL of a solution of CellTiter 96 Aqueous One Solution (Promega, Madison, Wis., USA) containing MTS [3-(4,5-dimethylthiazol-2-yl)-5-(3-carboxymethoxyphenyl)-2-(4-sulfophenyl)-2H-tetrazolium)] and an electron coupling reagent (phenazine ethosulfate) were added to each well with an auto-repeat pipetor. The plates were incubated for 3 hours during which time the reagent was bio-reduced into a colored formazan product by the intracellular dehydrogenase enzymes of metabolically active cells. The quantity of formazan produced is directly proportional to the number of living cells in the cultures. The absorbance (A) was measured at 490 nm (reference at 690 nm) on a PowerWave 200 microplate reader (Bio-Tek Instruments). The results were expressed as percent cell viability, calculated for each drug concentration using the formula:

% Viability=(A treated group/A control group)×100%.

(100% viability was represented by samples with DMSO vehicle control).

The half-maximal inhibitory concentration, IC₅₀, values were obtained from the sigmoidal curve by plotting the percentage viability of cells against the drug concentration using Psi-Plot version 9, Poly Software International (Pearl River, N.Y., USA).

### NMR Experimental

NMR spectra were acquired at 25° C. using a Bruker DRX-600 spectrometer with a Nalorac 5 mm inverse H/C/N probe with a z-axis gradient. 2D DQF-COSY, edited decoupled HSQC and magnitude mode HMBC experiments used standard Bruker pulse sequences. All 2D acquisitions used 750 t₁ increments and data were zero-filled to give a 2048 (F₂)×1024 (F₁) point 2D matrix. NMR data was processed and analyzed using the Felix software package. 3D molecular modeling and energy minimization were performed using the Insight II (Accelerys, Inc.) software package.

Although a few embodiments have been shown and described, it would be appreciated by those skilled in the art that changes may be made in these embodiments without departing from the principles and spirit of the invention, the scope of which is defined in the claims and their equivalents.

What is claimed is:

1. A biologically active composition to treat cancer, comprising:
an extract of Schisandra chinensis; and
a pharmaceutical formulation,
wherein the composition is useful for treating colorectal cancer.

2. The composition of claim 1, wherein the extract comprises essentially at least one compound from the group consisting of gomisin A, angeloylgomisin H, deoxyschisandrin and gomisin N.

3. The composition of claim 1, wherein the extract comprises essentially gomisin N.

4. The composition of claim 1, wherein the extract exhibits an inhibitory activity against a human colon HT-29 cell line.

5. The composition of claim 4, wherein the inhibitory activity is expressed as a half-maximal inhibitory concentration (IC₅₀) in the range of 716 μM to 43 μM.

6. The composition of claim 1, wherein the pharmaceutical formulation is a member selected from the group consisting of a pill form, a tablet form, a capsule form, a liquid solution form, a liquid suspension form, a powder form, an intravenous solution form, a gel form and a suppository form.

7. A method of preparing a biologically active composition from Schisandra chinensis, comprising:
grinding dried berries of Schisandra chinensis into a powder of dried berries;
extracting the powder of dried berries with hexane to form a marc having been washed with hexane;
extracting the marc with dichloromethane to form a dichloromethane soluble fraction;
extracting the dichloromethane soluble fraction with ethyl acetate to form a dichloromethane-ethyl acetate soluble fraction;
removing fatty materials in the dichloromethane-ethyl acetate soluble fraction with hexane to yield a total hexane insoluble material extract;
purifying from the total hexane insoluble material extract by using analytical high performance liquid chromatography equipped with automated repeat injection/collection cycles and guided by a biological activity against colon cancer to form an extract of Schisandra chinensis; and
combining the extract of Schisandra chinensis with a pharmaceutical formulation to form the biologically active composition.

8. The method of preparing of claim 7, wherein the extract of Schisandra chinensis is essentially at least one member from the group consisting of gomisin A, angeloylgomisin H, deoxyschisandrin and gomisin N.

9. The method of preparing of claim 7, wherein the extract of Schisandra chinensis is essentially gomisin N.

10. The method of preparing of claim 7, wherein the biological activity against colorectal cancer is indicated by an inhibitory activity of a human colon HT-29 cell line.

11. The method of preparing of claim 10, wherein the inhibitory activity is expressed as a half-maximal inhibitory concentration (IC₅₀) in the range of 716 μM to 43 μM.

12. The method of preparing of claim 7, wherein the pharmaceutical formulation is a member selected from the group consisting of a pill form, a tablet form, a capsule form, a liquid solution form, a liquid suspension form, a powder faun, an intravenous solution form, a gel form and a suppository form.

13. A method of treating colorectal cancer, comprising: administering a biologically active composition of Schisandra chinensis to a person having symptoms of colorectal cancer, wherein the composition comprises an extract of Schisandra chinensis and a pharmaceutical formulation.

14. The method of treating of claim 13, wherein the extract comprises essentially at least one compound from the group consisting of gomisin A, angeloylgomisin H, deoxyschisandrin and gomisin N.

15. The method of treating of claim 13, wherein the extract comprises essentially gomisin N.

16. The method of treating of claim 13, wherein the extract exhibits an inhibitory activity against a human colon HT-29 cell line.

17. The method of treating of claim 16, wherein the inhibitory activity is expressed as a half-maximal inhibitory concentration (IC₅₀) in the range of 716 μM to 43 μM.

18. The method of treating claim 13, wherein the pharmaceutical formulation is a member selected from the group consisting of a pill form, a tablet form, a capsule form, a liquid solution form, a liquid suspension form, a powder form, an intravenous solution form, a gel form and a suppository form.
